(19) United States
(12) Patent Application Publication
(10) Pub. No.: US 2006/0235076 A1
(43) Pub. Date: Oct. 19, 2006

(54) Medicinal composition for treating infection with drug-resistant staphylococcus aureus

(75) Inventors: Tomihiko Higuchi, Naruto-shi (JP); Hirofumi Shibata, Itano-gun (JP); Yoichi Sato, Tokushima-shi (JP); Nobuhisa Takaishi, Tokushima-shi (JP); Kazuyoshi Kawazoe, Naruto-shi (JP); Kotaro Murakami, Kumamoto-shi (JP)

Correspondence Address: BACON & THOMAS, PLLC, 625 SLATERS LANE, FOURTH FLOOR, ALEXANDRIA, VA, 22314, US

(73) Assignees: ALPS PHARMACEUTICAL IND. CO., LTD., Furukawa-cho, Hida-shi (JP); MicroBiotech Inc., Naruto-cho, Naruto-shi (JP)

(21) Appl. No.: 10/543,336

(22) PCT Filed: Jan. 28, 2004
(86) PCT No.: PCT/JP04/00751

§ 371 Date: May 8, 2006

(30) Foreign Application Priority Data
Jan. 29, 2003 (JP) 2003-020611

(51) Int. Cl. A61K 31/235

(52) U.S. Cl. 514/543

(57) ABSTRACT

The invention relates to a therapy of an infection with a drug resistant bacterium wherein a characteristic of multivalent phenol derivatives and/or extracts from “Tara” which enhance the activity of β-lactam antibiotics on the drug resistant bacteria is utilized. Specifically, the invention relates to an enhancer of β-lactam antibiotics comprising a multivalent phenol derivative or its pharmaceutically acceptable salt, a pharmaceutical composition comprising a β-lactam antibiotic and a multivalent phenol derivative and/or extract from “Tara” for the therapy of an infection with a drug resistant bacterium, a disinfectant for the same, and a functional food comprising a multivalent phenol derivative and/or extract from “Tara”.

### TECHNICAL FIELD

The invention relates to a pharmaceutical composition for the therapy of an infection with a drug-resistant bacterium wherein a characteristic of multivalent phenol derivatives and/or extracts from “Tara” which enhance the activity of β-lactam antibiotics on the drug resistant bacteria is utilized. Furthermore, the invention relates to a disinfectant or a functional food having antibacterial activity on resistant bacteria.

### BACKGROUND OF THE INVENTION

Penicillin, which is the first antibiotic, has a β-lactam ring, and has exerted an excellent efficacy toward Staphylococci. However, penicillin resistant bacteria which produce an enzyme, penicillinase (β-lactamase), that degrades penicillin has emerged.

In regard to these penicillin resistant bacteria, almost all problems have appeared to be solved in clinical aspects by research and development of penicillinase resistant penicillin such as methicillin and cephems antibiotics. But, a year after methicillin was developed, a strain of MRSA emerged which is resistant to it, and then MRSA which is resistant to all of the antibiotics is clinically isolated.

MRSA have resulted in critical social problems as multiple drug resistant Staphylococcus aureus having broad resistance to not only penicillin antibiotics but also cephem antibiotics and aminoglycoside, macrolide, and new quinolone antibiotics.

At present, vancomycin (VCM) etc. is used as antibiotics for MRSA infections, however, short term bactericidal action of VCM is not so potent and VCM is involved in problems of serious side effects such as auricular toxicity and renal toxicity. Therefore, the development of novel antibacterial drugs which are effective on such resistant bacteria is required as an urgent matter.

### DISCLOSURE OF THE INVENTION

While searching compounds which have anti-MRSA activities among Chinese herbal medicine exhibiting no or weak side effects, the inventors found an interesting fact that extract of “Tara” (Caesalpinia spinosa) and multivalent phenol derivatives obtained from the extract suppress the resistance against β-lactam agents, and induce the sensitivity. The present invention was accomplished on the basis of such findings.

The “Tara” in the invention is Caesalpinia spinosa, leguminosae, a native of Peru, which is different from “Taranoki” in Japan, and is known to contain ellagic acid which prevents spots and freckles due to sunburn (e.g., LION Life Information, “How to prevent spots and freckles due to sunburn”, Internet, searched on Nov. 6, 1992, <http://www.lion.co.jp/life/life3p2.htm>). Also, it is described that gallotannin extracted from “Tara” showed deodorization activity (e.g., Kokai publication No. 09-327504).

However, it is not known that extract from “Tara” have enhancing activities on the resistant bacteria by combinations of β-lactam antibiotics.

First aspect of the invention is an enhancer of β-lactam antibiotics comprising a multivalent phenol derivative or its pharmaceutically acceptable salt.

Second aspect of the invention is an enhancer of β-lactam antibiotics comprising extract from “Tara” which comprises a multivalent phenol derivative or its pharmaceutically acceptable salt as an active ingredient.

In the invention, “Tara” means Caesalpinia spinosa, leguminosae, a native of Peru, containing about 0.25% of multivalent phenol derivatives in the whole plant. Extracts from “Tara” means products extracted with a suitable organic solvent or water.

An organic solvent is e.g., methanol or ethanol, and their mixture with water is allowable. Any part of “Tara” can be used. Usually, 50% ethanol extract from “Tara” contains about 0.32% of multivalent phenol derivatives. Various gallates from commercial sources can be used in the invention.

Preferable β-lactam antibiotics are oxacillin, cefapirin, ampicillin, penicillin, and cefoxitin; oxacillin, and cefapirin are more preferable.

Multivalent phenol derivatives includes a derivative of the formula I, 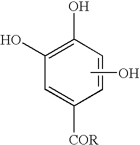
wherein R is a lower alkyl, OR¹ (R¹ is a lower alkyl), or a catechin anion.

More specifically, a multivalent phenol derivative includes a gallic acid derivative of the formula II, 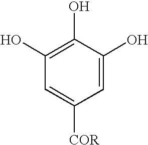
wherein R is OR¹ (R¹ is a lower alkyl), or catechin anion, and e.g., methyl gallate, ethyl gallate, n-propyl gallate, n-butyl gallate, n-pentyl gallate, n-hexyl gallate, n-heptyl gallate, n-octyl gallate, n-nonyl gallate, n-decyl gallate, n-undecyl gallate, n-lauryl gallate, isobutyl gallate, Isoamyl gallate, catechin gallate, gallocatechin gallate, and epicatechin gallate are included. More preferably, n-propyl gallate, n-pentyl gallate, isoamyl gallate, and catechin gallate are included.

In addition, quinic acid derivatives in which OH-substituents are esterified with gallic acids, i.e., methyl 4,5-digalloylquinate, 3,4,5-trigalloylquinic acid, methyl 3,4,5-trigalloylquinate, and 3,4-digalloylquinic acid were found in the “Tara” extracts. These derivatives also have enhancing activity of β-Lactam antibiotics and especially, 3,4,5-trigalloylquinic acid and methyl 4,5-digalloylquinate are excellent.

Third aspect of the invention is a pharmaceutical composition for the therapy of an infection with a drug resistant bacterium comprising a β-lactam antibiotic and the enhancer described above.

Fourth aspect of the invention is a disinfectant disinfectant having antibacterial activity on resistant bacteria comprising a β-lactam antibiotic and “Tara” extract and/or a multivalent phenol derivative as an active ingredient.

Fifth aspect of the invention is a functional food for the prevention and/or improvement of an infection with a drug resistant bacterium comprising a multivalent phenol derivative and/or “Tara” extract, which enhance antibacterial activity of an antibiotic on resistant bacteria.

### BEST MODE FOR CARRYING OUT THE INVENTION

The term “Lower alkyl” in the invention refers to saturated straight or branched hydrocarbon chain having 1 to 12 carbon atoms. For example, methyl, ethyl, n-propyl, n-butyl, n-pentyl, n-hexyl, n-heptyl, n-octyl, n-nonyl, n-decyl, n-undecyl, n-lauryl, isobutyl, and isoamyl are included. The term “cathechin anion” refers to e.g., anion of catechin, gallocatechin, or epicatechin.

Multivalent phenol derivatives, antibiotics and antibacterial agents in the invention include pharmaceutically acceptable salts of them. Pharmaceutically acceptable salts refer to medicinally allowable salts commonly used, e.g., salts of sodium, potassium or calcium, or acid-additive salts such as amine salts(e.g., a dibenzylamine salt) and HCl salt. Also, other active ingridient may be included in the invention.

Examples of drug resistant bacteria include methicillin resistant Staphylococcus aureus (MRSA), penicillinase producing Staphylococcus aureus, vancomycin resistance Enterococcus (VRE), vancomycin resistance Staphylococcus aureus (VRSA), penicillin resistance Streptococcus pneumoniae (PRSP), substrate specificity expanded β-lactamase (ESBLSs), and the like. The drug resistant bacterium is preferably MRSA, and may be penicillinase producing Staphylococcus aureus.

Examples of the β-lactam antibiotics used in the invention include benzylpenicillin, phenoxymethylpenicillin, phenethicillin, propicillin, ampicillin, methicillin, oxacillin, cloxacillin, flucloxacillin, dicloxacillin, hetacillin, talampicillin, bacampicillin, lenampicillin, amoxicillin, ciclacillin, carbenicillin, sulbenicillin, ticarcillin, carindacillin, carfecillin, piperacillin, mezlocillin, aspoxicillin, cephaloridine, cefazolin, cefapirin, cephacetrile, ceftezole, cephaloglycin, cephalexin, cefatrizine, cefaclor, cefroxadine, cefadroxil, cefamandole, cefotiam, cephalothin, cephradine, cefuroxime, cefoxitin, cefotaxime, ceftizoxime, cefmenoxime, cefodizime, ceftriaxone, cefuzonam, ceftazidime, cefepim, cefpirome, cefozopran, cefoselis, cefluprenam, cefoperazone, cefpimizole, cefpiramide, cefixime, cefteram pivoxil, cefpodoxime proxetil, ceftibuten, cefetamet pivoxil, cefdinir, cefditoren pivoxil, cefcapene pivoxil, cefsulodin, cefoxitin, cefmetazole, latamoxef, cefotetan, cefbuperazone, cefminox, flomoxef, aztreonam, carumonam, imipenem, panipenem, meropenem, viapenem, faropenem, ritipenem acoxil, or mixtures thereof. The β-lactam antibiotics are preferably ampicillin, benzylpenicillin, phenethicillin, methicillin, oxacillin, carbenicillin, cefapirin, cefradine, cefuroxime, cefoxitin, cefotaxime, and panipenem; more preferably, ampicillin, cefapirin, benzylpenicillin, oxacillin, cefoxitin or mixtures thereof.

The antibiotics may be in the form of a pharmaceutically acceptable salt. Pharmaceutically acceptable salts refer to salts which are allowable in medical aspects used in general as salts of an antibiotic, including for example, salts of sodium, potassium, calcium and the like, and amine salts of procaine, dibenzylamine, ethylenediamine, ethanolamine, methylglucamine, taurine, and the like, as well as acid addition salts such as hydrochlorides, and basic amino acids and the like.

Mode of administration of the multivalent phenol derivatives and/or extracts from “Tara” of the invention includes parenteral administration, oral administration or topical administration similarly to the case of conventional antibiotics. In general, the administration in an injectable is suitable. In this instance, the injectable is prepared by the conventional process, which also involves the cases in which the compound is dissolved in an adequate vehicle, e.g., sterilized distilled water, saline and the like, to give an injectable form.

Moreover, the multivalent phenol derivatives and/or extracts from “Tara” can be orally administered by the combination with a -lactam antibiotics in a variety of dosage forms. Examples of the dosage form include for example, tablet, capsule, sugarcoated tablet or the like, liquid solution or suspension.

Total dose of both agents of the multivalent phenol derivative and the β-lactam antibiotic may vary depending on types of the combined agent, the ratio of the combined use, or the age, body weight, symptoms of the patient and the route for administration. For example, when administered to an adult (body weight: about 50 kg), 10 mg-2 g in total weight of both agents combined per single dosage is administered from once to three times per a day. To achieve the best therapeutic effects may be intended by altering the dose and route for administration.

In accordance with the invention, it is possible to apply a wide range of weight ratio of the multivalent phenol derivative and the β-lactam antibiotic which are used in combination or admixed together. Further, because the ratio of the combined use varies depending on types and severity of the infection, and types of the β-lactam antibiotic used in combination, the ratio of the combined use is not particularly limited. Accordingly, combination of the concentration by which effects through the combined use can be expected is achieved upon combination in the range of usual dosages.

The pharmaceutical composition of the invention is usually prepared according to the conventional process, and is administered in a pharmaceutically adequate form. For example, solid peroral form may include a diluent such as lactose, dextrose, saccharose, cellulose, and cornstarch and potato starch, a lubricant such as silica, talc, stearic acid, magnesium stearate or calcium stearate, and/or polyethyleneglycol, a binder such as starch, gum arabic, gelatin, methyl cellulose, carboxymethylcellulose, polyvinyl pyrrolidine, a disintegrant such as starch, alginic acid, alginate, glycolic acid starch sodium, a foaming agent, a pigment, an edulcorant, a wetting agent such as lecithin, polysorbate, lauryl sulfate, and a pharmaceutically inactive substance which is generally nontoxic and used for a pharmaceutical formulation, in addition to the active compound.

The above-described pharmaceutical composition is produced by a known process such as for example, mixing, granulation, and manufacture of tablets, sugar coating, or coating process.

In a case of parenteral administration, suppository to which rectal application is intended is also feasible, however, frequently used dosage form is an injectable. The injectable includes dosage forms having different appearances such as liquid formulations, formulations for dissolution before use, and suspension formulations, which are fundamentally identical in respect of requiring sterilization of the active ingredient by an appropriate method, followed by directly placing into a vessel, and sealing.

Most convenient formulation process includes a process in which the active ingredient is sterilized by an appropriate method, thereafter separately, or after being physically mixed, the aliquot thereof is separately formulated. Further, when a liquid dosage form is selected, a process can be applied in which the active ingredient is dissolved in an appropriate medium, followed by sterilization by filtration, filling in an appropriate ampoule or vial, and sealing.

In this case, frequently used media include distilled water for injection, which is not limited thereto in accordance with the invention. Additionally, additives such as soothing agents having a local anesthetic action such as procaine hydrochloride, xylocaine hydrochloride, benzyl alcohol and phenol, antiseptic agents such as benzyl alcohol, phenol, methyl or propylparaben and chlorobutanol, buffer agents such as a sodium salt of citric acid, acetic acid, phosphoric acid, auxiliary agents for the dissolution such as ethanol, propylene glycol, arginine hydrochloride, stabilizing agents such as L-cysteine, L-methionine, L-histidine, as well as isotonizing agents can be also added, if required.

The multivalent phenol derivative and/or extractions from “Tara” of the invention can be prepared as an antibacterial agent or a bactericidal agent. The antibacterial agent or the bactericidal agent is comprising about 0.1-10% in weight of multivalent phenol derivative and/or extract from “Tara”, and a suitable amount of β-lactam antibiotics. Other antibacterial agent or bactericidal agent is also included. These antibacterial agents or bactericidal agents are used to disinfect instruments such as scissors, scalpels, catheters, as well as excrements of patients, and to irrigate skins, mucosa and wounds.

The enhancer of the invention can be applied as a form of a functional food to prevent an infection with a drug resistant bacteria.

A form of the invention when used as a food is not limited and for example, a drink, a solid product, and jellied food are included; in the solid product a processed form as a preparation of powder, granule, tablet and capsule is included. The multivalent phenol derivative and/or “Tara” extract may be contained in a noodle like udon (a wheat noodle) or soba(a buckwheat noodle), a cookie, a biscuit, a candy, bread, a cake, and other foods; it may be added in drinks such as a carbonated drink, lactic acid drink etc.

### TEST EXAMPLES

### Test Example 1

### Enhancing Activity of “Tara” Extract for Oxacillin (Disk Diffusion Method)

After extraction of 10 g of “Tara” pod with 100 ml of 50% methanol and filtration, “Tara” extract was obtained. The disk diffusion method was used to measure the antibacterial activity. As a base medium, Mueller-Hinton Agar (MHA) and Oxacillin-containing (10 μg/ml) MHA were prepared, and they were covered with semifluid MHA which was pre-incubated with MRSA No. 5, 1×10⁵ CFU/ml. Disks having a diameter of 6 mm were placed, and 100 μg of “Tara” extract was added on them. They were incubated at 37° C. and the diameters of the zones of inhibition around the disks were measured after 24 hr and 48 hr.

Result

TABLE 1

| | Diameter of the zone of inhibition (mm) | | | | |
|---|---|---|---|---|---|
| | 24 hr | | 48 hr | | |
| Name | Control | Oxacillin | Control | Oxacillin | Difference ^{a)} |
| “Tara” extract | 21 | 24 | 21 | 24 | 3 |

^{a)} Difference of the diameters of inhibition zone between Oxacillin and Control

As shown in Table 1, the diameter of oxacillin-containing disk was 24 mm and increased by 3 mm, while the diameter of Control was 21 mm. It means the sensitivity of the antibiotic was elevated by ingredients in “Tara” extract.

### Test Example 2

### Enhancing Activity for the Antibacterial Activity of Oxacillin by Various Gallates (by the Agar Plate Dilution Method)

According to the agar plate dilution method defined by Japan Society of Chemotherapy, MIC (Minimum inhibitory concentration) was determined. CAMHA was prepared by adding 50 mg/l of Ca-ion, 25 mg/l of Mg-ion, and 2% of NaCl to Mueller-Hinton II Agar (MHA), and it was used as a medium for use in the measurement of sensitivity. The bacterial liquid was incubated at 37° C. overnight and diluted to 10⁶ CFU/ml with saline. To the plate for use in the measurement of sensitivity was seeded the bacterial liquid with a micro planter (Sakuma Seisakusho), and MIC was determined after incubation at 37° C. for 24 hr. The results were shown in Table 2-1 and 2-2.

TABLE 2-1
Oxacillin MIC (μg/ml)

| | | | Methyl | | Ethyl | | Propyl | | Butyl | |
|---|---|---|---|---|---|---|---|---|---|---|
| | | | gallate | | gallate | | gallate | | gallate | |
| Strain | | | 25 | 50 | 25 | 50 | 25 | 50 | 25 | 50 |
| No. | | — | μg/ml | μg/ml | μg/ml | μg/ml | μg/ml | μg/ml | μg/ml | μg/ml |
| MRSA | 1 | >256 | >256 | >256 | 256 | >256 | 256 | 128 | 128 | 16 |
| | 2 | 256 | 64 | 8 | 4 | 0.25 | 2 | 0.063 | 4 | ND |
| | 3 | 128 | 32 | 8 | 8 | 8 | 8 | 2 | 4 | 0.5 |
| | 4 | 256 | 256 | 64 | 128 | <0.016 | 128 | <0.016 | 64 | ND |
| | 5 | 256 | 64 | 16 | 16 | 8 | 8 | 1 | 8 | <0.016 |
| | 6 | >256 | >256 | 25 | 256 | 1 | 256 | <0.016 | 64 | ND |
| | 7 | >256 | 128 | 16 | 32 | 8 | 32 | 2 | 16 | <0.016 |
| | 8 | 128 | 128 | 64 | 32 | 16 | 16 | 2 | 16 | ND |
| | 9 | 256 | 128 | ND | 16 | ND | 8 | ND | 16 | <0.016 |
| | 10 | 128 | 2 | 2 | 1 | <0.016 | 0.5 | ND | <0.5 | ND |
| | 12 | >256 | 256 | 256 | 64 | 256 | 16 | 16 | 64 | 8 |
| | 16 | 256 | 256 | 256 | 128 | 16 | 128 | 2 | 128 | ND |
| | 17 | 256 | 128 | 4 | 64 | <0.016 | 32 | 1 | 32 | 0.25 |
| | 18 | >256 | 256 | 32 | 128 | 0.13 | 128 | 0.125 | 64 | <0.016 |
| | 20 | 64 | 4 | 8 | 2 | 4 | 2 | 1 | 1 | 0.5 |
| | 21 | 64 | 16 | 8 | 4 | 8 | 2 | 2 | 2 | 1 |
| | 22 | 32 | 16 | 16 | 8 | 8 | 2 | 4 | 2 | 2 |
| | 24 | <0.5 | <0.5 | 1 | <0.5 | 0.5 | 0.5 | 0.5 | <0.5 | 0.25 |
| | COL | 256 | 128 | 64 | 64 | 32 | 32 | 8 | 32 | 4 |
| MSSA | 1003 | 0.25 | <0.5 | 1 | 1 | 0.5 | 0.5 | 0.5 | <0.5 | 0.5 |
| | 1010 | 0.25 | 1 | 2 | 1 | 1 | 1 | 1 | 1 | 0.5 |
| | 1020 | 1 | <0.5 | 1 | 1 | 1 | 0.25 | 0.25 | <0.5 | 0.13 |
| | 1023 | 2 | 1 | 1 | 1 | 1 | 1 | 1 | <0.5 | 0.5 |
| | 1029 | 4 | 1 | 1 | 1 | 1 | 1 | 1 | 1 | 1 |
| | 1032 | 0.13 | <0.5 | 1 | 1 | 1 | 0.5 | 0.5 | <0.5 | 0.25 |
| ATCC | 6538 | 0.063 | <0.5 | 0.25 | <0.5 | 0.25 | 0.13 | 0.25 | <0.5 | 0.13 |

| | | | | | | | | | |
|---|---|---|---|---|---|---|---|---|---|
| RN4220 | 0.13 | <0.5 | 0.25 | <0.5 | 0.25 | 0.25 | 0.125 | <0.5 | ND |

| | | |
|---|---|---|
| Pentyl | Hexyl | Heptyl |
| gallate | gallate | gallate |

| Strain | | 12.5 | 25 | 12.5 | 25 | 12.5 | 25 |
|---|---|---|---|---|---|---|---|
| No. | | μg/ml | μg/ml | μg/ml | μg/ml | μg/ml | μg/ml |
| MRSA | 1 | 256 | 64 | >256 | 128 | 256 | >256 |
| | 2 | 8 | 0.063 | 64 | 0.031 | 8 | ND |
| | 3 | 8 | 0.25 | 64 | 2 | 64 | 2 |
| | 4 | 128 | 2 | >256 | 16 | 256 | ND |
| | 5 | 64 | 2 | 64 | 4 | 128 | 32 |
| | 6 | 32 | 1 | 256 | 2 | 256 | ND |
| | 7 | 32 | 0.13 | 256 | 2 | 256 | 32 |
| | 8 | 16 | 0.13 | 64 | 2 | 128 | 32 |
| | 9 | 128 | 0.25 | >256 | 2 | 256 | ND |
| | 10 | 4 | 0.13 | ND | ND | 0.5 | 0.25 |
| | 12 | 128 | 16 | 256 | 16 | 128 | 256 |
| | 16 | 128 | 2 | 256 | 4 | 256 | 64 |
| | 17 | 64 | 0.25 | 256 | 4 | 128 | 2 |
| | 18 | 128 | 2 | 256 | 4 | 256 | 2 |
| | 20 | 1 | 0.5 | 8 | 1 | 8 | 2 |
| | 21 | 16 | 2 | 8 | 4 | 16 | 32 |
| | 22 | 8 | 1 | 1 | 4 | 16 | 32 |
| | 24 | 0.5 | 0.25 | 1 | 1 | 0.5 | 0.13 |
| | COL | 32 | 2 | 256 | 32 | 64 | 32 |
| MSSA | 1003 | 0.25 | 0.031 | 0.25 | 0.031 | 0.13 | ND |
| | 1010 | 0.25 | 0.25 | 0.5 | 0.031 | 0.25 | ND |
| | 1020 | 0.5 | 0.25 | 1 | 0.031 | 0.5 | ND |
| | 1023 | 1 | 0.5 | 1 | 1 | 0.5 | 0.25 |
| | 1029 | 2 | 1 | 2 | 1 | 0.5 | 0.25 |
| | 1032 | 0.25 | 0.25 | 0.5 | 0.13 | 0.25 | 0.25 |
| ATCC | 6538 | 0.13 | 0.13 | 0.25 | 0.031 | 0.063 | ND |

| | | | | | | |
|---|---|---|---|---|---|---|
| RN4220 | 0.13 | 0.063 | 0.13 | 0.13 | 0.063 | ND |

ND: not determine

TABLE 2-2
Oxacillin MIC (μg/ml)

| | | Octyl | | Nonyl | | Decyl | | Undecyl | | Lauryl | | isoButyl | | isoAmyl | |
|---|---|---|---|---|---|---|---|---|---|---|---|---|---|---|---|
| | | gallate | | gallate | | gallate | | gallate | | gallate | | gallate | | gallate | |
| | | 12.5 | 25 | 12.5 | 25 | 12.5 | 25 | 12.5 | 25 | 12.5 | 25 | 25 | 50 | 12.5 | 25 |
| Strain No. | | μg/ml | μg/ml | μg/ml | μg/ml | μg/ml | μg/ml | μg/ml | μg/ml | μg/ml | μg/ml | μg/ml | μg/ml | μg/ml | μg/ml |
| MRSA | 1 | 256 | ND | 256 | ND | >256 | ND | >256 | ND | 256 | >256 | 256 | 32 | >256 | 64 |
| | 2 | 0.13 | ND | 0.13 | ND | 32 | ND | 16 | ND | 256 | >256 | 8 | ND | 128 | 0.5 |
| | 3 | 8 | ND | 128 | ND | 128 | ND | 256 | ND | 128 | 256 | 4 | 0.5 | 128 | 0.063 |
| | 4 | 2 | ND | 2 | ND | 16 | ND | >256 | ND | 256 | 256 | 128 | 1 | 256 | 8 |
| | 5 | 64 | ND | 0.13 | ND | 1 | ND | 256 | ND | 256 | 256 | 16 | 0.25 | 128 | 0.063 |
| | 6 | 64 | ND | 0.063 | ND | 0.063 | ND | >256 | ND | 256 | >256 | 128 | ND | 128 | 1 |
| | 7 | 64 | ND | 128 | ND | 128 | ND | >256 | ND | 256 | >256 | 16 | ND | 256 | <0.063 |
| | 8 | 128 | ND | 256 | ND | 256 | ND | >256 | ND | 128 | 256 | 16 | 1 | 256 | 0.063 |
| | 9 | 1 | ND | 0.5 | ND | 128 | ND | 256 | ND | 256 | 256 | 128 | ND | 256 | 4 |
| | 10 | 32 | ND | ND | ND | ND | ND | ND | ND | 8 | 4 | <0.5 | ND | 32 | ND |
| | 12 | 128 | ND | 256 | ND | 256 | ND | 256 | ND | 256 | >256 | 128 | 8 | 256 | 4 |
| | 16 | 128 | ND | 1 | ND | 2 | ND | 256 | ND | 256 | 256 | 256 | 1 | 256 | 4 |
| | 17 | 1 | ND | 0.5 | ND | 2 | ND | 256 | ND | 256 | 256 | 128 | 1 | 128 | 4 |
| | 18 | 4 | ND | 0.13 | ND | 16 | ND | >256 | ND | 256 | >256 | 128 | 1 | 256 | 16 |
| | 20 | 16 | ND | 4 | ND | 4 | ND | 8 | ND | 8 | 64 | 1 | 0.5 | 4 | 0.25 |
| | 21 | 16 | ND | 32 | ND | 16 | ND | 32 | ND | 32 | 64 | 4 | 1 | 32 | 0.5 |
| | 22 | 64 | ND | 32 | ND | 64 | ND | 128 | ND | 32 | 128 | 4 | 2 | 32 | 1 |
| | 24 | 0.13 | ND | 0.063 | ND | 0.063 | ND | 0.5 | ND | <0.5 | 0.5 | 1 | 0.25 | 1 | 0.5 |
| | COL | 128 | ND | 0.063 | ND | 0.13 | ND | 256 | ND | 256 | 256 | 64 | 4 | 128 | 2 |
| MSSA | 1003 | 0.13 | ND | 0.063 | ND | 0.13 | ND | 0.063 | ND | <0.5 | 0.13 | <0.5 | 0.5 | 0.25 | 0.25 |
| | 1010 | 0.13 | ND | 0.13 | ND | 0.13 | ND | 0.25 | ND | <0.5 | 2 | 1 | 0.5 | 0.25 | 0.25 |
| | 1020 | 0.13 | ND | 0.063 | ND | 0.063 | ND | 0.25 | ND | 1 | 2 | <0.5 | 0.13 | 1 | 0.25 |
| | 1023 | 0.13 | ND | 0.063 | ND | 0.063 | ND | 0.5 | ND | 2 | 2 | 1 | 0.5 | 2 | 0.25 |
| | 1029 | 0.13 | ND | 0.063 | ND | 0.13 | ND | 0.5 | ND | 1 | 0.5 | 1 | 0.5 | 4 | 0.5 |
| | 1032 | 0.13 | ND | 0.063 | ND | 0.063 | ND | 0.13 | ND | <0.5 | 0.5 | <0.5 | 0.25 | 0.25 | 0.25 |
| ATCC | 6538 | 0.13 | ND | 0.063 | ND | 0.063 | ND | 0.13 | ND | <0.5 | 0.13 | <0.5 | 0.25 | 0.13 | 0.13 |

| | | | | | | | | | | | | | | |
|---|---|---|---|---|---|---|---|---|---|---|---|---|---|---|
| RN4220 | 0.063 | ND | 0.063 | ND | 0.063 | ND | 0.13 | ND | <0.5 | 0.13 | <0.5 | 0.063 | 0.13 | 0.063 |

ND: not determine

Result

With respect to the effect of combination use with oxacillin, the effect was potentiated as the number of carbon atoms in the side chain of the multivalent phenol was increased, and MIC for oxacillin with MRSA No. 2 was elevated to 0.06 μg/ml from 256 μg/ml by 25 μg/ml of n-pentyl gallate. But the effect of the combination use was decreased when the number of carbon atom was further increased, and C4-C6 of the side chain was found to show the most potential effect of the combination use. These effects were found in MSSA as well as in MRSA. Furthermore, isobutyl gallate and isoamyl gallate, the side chains of which were branched, also have the potent effects of the combination use.

In addition, MIC for oxacillin alone with MRSA Strain No. 2 is 256, MIC for n-pentyl gallate alone is 67.5 as shown in Table3-1 below; however, MIC for oxacillin was elevated to 0.063 when combined with 25 μg/ml of n-pentyl gallate showing no antibacterial effect, and n-pentyl gallate is found to have excellent potentiating effect.

### Test Example 3

### Antibacterial Activity of Gallic Acid and Various Gallates Alone (by the Agar Plate Dilution Method)

MIC for gallic acid, various gallates and multivalent phenol derivatives alone with MRSA and MSSA were determined as described in experiment 2. The results were shown in Table 3-1 and 3-2.

TABLE 3-1

| | Gallic | Methyl | Ethyl | Propyl | Butyl | Pentyl | Hexyl | Heptyl | Octyl | Nonyl | Decyl |
|---|---|---|---|---|---|---|---|---|---|---|---|
| Name | acid | gallate | gallate | gallate | gallate | gallate | gallate | gallate | gallate | gallate | gallate |
| MRSA #1 | 31.3 | 250 | 125 | 125 | 125 | 62.5 | 62.5 | 31.3 | 31.3 | 15.7 | 15.7 |
| MRSA #2 | 62.5 | 125 | 62.5 | 62.5 | 125 | 62.5 | 31.3 | 31.3 | 31.3 | 15.7 | 15.7 |
| MRSA #3 | 31.3 | 250 | 125 | 125 | 125 | 62.5 | 31.3 | 31.3 | 31.3 | 15.7 | 15.7 |
| MRSA #4 | 62.5 | 125 | 62.5 | 125 | 125 | 62.5 | 31.3 | 31.3 | 31.3 | 15.7 | 15.7 |
| MRSA #5 | 31.3 | 250 | 125 | 125 | 125 | 62.5 | 31.3 | 31.3 | 31.3 | 15.7 | 15.7 |
| MRSA #6 | 31.3 | 125 | 62.5 | 62.5 | 62.5 | 62.5 | 31.3 | 31.3 | 31.3 | 15.7 | 15.7 |
| MRSA #7 | 62.5 | 250 | 125 | 125 | 125 | 62.5 | 31.3 | 31.3 | 31.3 | 15.7 | 15.7 |
| MRSA #8 | 31.3 | 250 | 125 | 125 | 125 | 62.5 | 31.3 | 31.3 | 31.3 | 15.7 | 15.7 |
| MRSA #9 | 31.3 | 62.5 | 62.5 | 62.5 | 62.5 | 62.5 | 31.3 | 31.3 | 31.3 | 15.7 | 15.7 |
| MRSA #10 | 31.3 | 62.5 | 62.5 | 62.5 | 62.5 | 62.5 | 31.3 | 31.3 | 15.6 | 15.7 | 15.7 |
| MRSA #12 | 62.5 | 250 | 125 | 125 | 125 | 62.5 | 62.5 | 31.3 | 31.3 | 15.7 | 15.7 |
| MRSA #16 | 31.3 | 125 | 125 | 125 | 125 | 62.5 | 62.5 | 31.3 | 31.3 | 15.7 | 15.7 |
| MRSA #17 | 62.5 | 125 | 62.5 | 62.5 | 125 | 62.5 | 62.5 | 31.3 | 31.3 | 15.7 | 15.7 |
| MRSA #18 | 62.5 | 125 | 62.5 | 62.5 | 125 | 62.5 | 62.5 | 31.3 | 31.3 | 15.7 | 15.7 |
| MRSA #20 | 62.5 | 250 | 125 | 125 | 125 | 62.5 | 62.5 | 31.3 | 31.3 | 15.7 | 15.7 |
| MRSA #21 | 62.5 | 250 | 125 | 125 | 125 | 62.5 | 62.5 | 31.3 | 31.3 | 15.7 | 31.3 |
| MRSA #22 | 62.5 | 250 | 125 | 125 | 125 | 62.5 | 62.5 | 31.3 | 31.3 | 15.7 | 31.3 |
| MRSA #24 | 62.5 | 250 | 125 | 125 | 125 | 62.5 | 62.5 | 31.3 | 31.3 | 15.7 | 31.3 |
| COL | 62.5 | 250 | 125 | 125 | 125 | 62.5 | 31.3 | 31.3 | 31.3 | 15.7 | 15.7 |
| MSSA #1003 | 62.5 | 250 | 125 | 125 | 125 | 62.5 | 62.5 | 31.3 | 31.3 | 15.7 | 15.7 |
| MSSA #1010 | 62.5 | 250 | 125 | 125 | 125 | 62.5 | 62.5 | 31.3 | 31.3 | 15.7 | 15.7 |
| MSSA #1020 | 62.5 | 250 | 125 | 125 | 125 | 62.5 | 62.5 | 31.3 | 31.3 | 15.7 | 15.7 |
| MSSA #1023 | 31.3 | 125 | 62.5 | 62.5 | 125 | 62.5 | 62.5 | 31.3 | 31.3 | 15.7 | 15.7 |
| MSSA #1029 | 31.3 | 62.5 | 62.5 | 62.5 | 62.5 | 62.5 | 62.5 | 31.3 | 31.3 | 15.7 | 15.7 |
| MSSA #1032 | 31.3 | 250 | 125 | 125 | 125 | 62.5 | 62.5 | 31.3 | 31.3 | 15.7 | 15.7 |
| ATCC6538 | 62.5 | 250 | 125 | 125 | 125 | 62.5 | 31.3 | 31.3 | 31.3 | 15.7 | 15.7 |
| RN4220 | 62.5 | 125 | 125 | 62.5 | 62.5 | 62.5 | 62.5 | 31.3 | 31.3 | 15.7 | 15.7 |
| MIC₅₀ | 62.5 | 250 | 125 | 125 | 125 | 62.5 | 62.5 | 31.3 | 31.3 | 15.7 | 15.7 |

TABLE 3-2

| | | | | | | 2′,4′,5′- | | | |
|---|---|---|---|---|---|---|---|---|---|
| | Undecyl | Lauryl | Cetyl | Isobutyl | Isoamyl | trihydroxy | 2′,3′,4′-trihydroxy- | Isoamyl 4- | n-Amyl 4- |
| Name | gallate | gallate | gallate | gallate | gallate | butyrophenone | acetophenone | hydroxybenzoate | hydroxybenzoate |
| MRSA #1 | 31.3 | 62.5 | >250 | 125 | 62.5 | 62.5 | 62.5 | 62.5 | 62.5 |
| MRSA #2 | 31.3 | 62.5 | 250 | 62.5 | 62.5 | 125 | 62.5 | 62.5 | 62.5 |
| MRSA #3 | 31.3 | 62.5 | >250 | 125 | 62.5 | 62.5 | 62.5 | 62.5 | 62.5 |
| MRSA #4 | 31.3 | 62.5 | >250 | 125 | 62.5 | 125 | 62.5 | 62.5 | 62.5 |
| MRSA #5 | 31.3 | 62.5 | >250 | 125 | 62.5 | 62.5 | 62.5 | 62.5 | 62.5 |
| MRSA #6 | 31.3 | 31.3 | 125 | 62.5 | 62.5 | 62.5 | 31.3 | 62.5 | 31.3 |
| MRSA #7 | 31.3 | 31.3 | >250 | 125 | 62.5 | 62.5 | 62.5 | 62.5 | 62.5 |
| MRSA #8 | 31.3 | 31.3 | >250 | 125 | 62.5 | 125 | 62.5 | 62.5 | 31.3 |
| MRSA #9 | 31.3 | 31.3 | >250 | 62.5 | 62.5 | 62.5 | 62.5 | 62.5 | 31.3 |
| MRSA #10 | 31.3 | 15.6 | 125 | 62.5 | 31.3 | 62.5 | 31.3 | 31.3 | 62.5 |
| MRSA #12 | 31.3 | 62.5 | >250 | 125 | 62.5 | 125 | 62.5 | 62.5 | 62.5 |
| MRSA #16 | 31.3 | 62.5 | >250 | 125 | 62.5 | 62.5 | 62.5 | 62.5 | 62.5 |
| MRSA #17 | 31.3 | 62.5 | >250 | 125 | 62.5 | 125 | 62.5 | 62.5 | 62.5 |
| MRSA #18 | 31.3 | 62.5 | >250 | 125 | 62.5 | 125 | 62.5 | 62.5 | 62.5 |
| MRSA #20 | 31.3 | 62.5 | >250 | 125 | 62.5 | 125 | 62.5 | 62.5 | 62.5 |
| MRSA #21 | 31.3 | 62.5 | >250 | 125 | 62.5 | 125 | 62.5 | 62.5 | 62.5 |
| MRSA #22 | 31.3 | 62.5 | >250 | 125 | 125 | 125 | 62.5 | 62.5 | 62.5 |
| MRSA #24 | 31.3 | 62.5 | >250 | 125 | 125 | 125 | 62.5 | 62.5 | 62.5 |
| COL | 31.3 | 31.3 | 250 | 125 | 62.5 | 125 | 62.5 | 62.5 | 62.5 |
| MSSA #1003 | 31.3 | 62.6 | >250 | 125 | 125 | 125 | 62.5 | 62.5 | 62.5 |
| MSSA #1010 | 31.3 | 62.5 | >250 | 125 | 62.5 | 125 | 62.5 | 62.5 | 62.5 |
| MSSA #1020 | 31.3 | 62.5 | >250 | 125 | 62.5 | 125 | 62.5 | 62.5 | 62.5 |
| MSSA #1023 | 31.3 | 62.5 | >250 | 125 | 62.5 | 62.5 | 31.3 | 62.5 | 31.3 |
| MSSA #1029 | 31.3 | 62.5 | 250 | 62.5 | 62.5 | 62.5 | 31.3 | 62.5 | 62.5 |
| MSSA #1032 | 31.3 | 62.5 | 250 | 125 | 62.5 | 62.5 | 62.5 | 62.5 | 62.5 |
| ATCC6538 | 31.3 | 62.5 | >250 | 125 | 62.5 | 125 | 62.5 | 62.5 | 62.5 |
| RN4220 | 31.3 | 62.5 | 250 | 125 | 62.5 | 125 | 62.5 | 62.5 | 62.5 |
| MIC₅₀ | 31.3 | 62.5 | >250 | 125 | 62.5 | 125 | 62.5 | 62.5 | 62.5 |

Result

As shown in Table 3, antibacterial activities of gallic acid and various gallates alone with MRSA and MSSR were potentiated as the number of carbon atoms was increased, and nonyl gallate and decyl gallate in the range of C9-C 10 were most potent and their MICs were 15.7 μg/ml. The activity was, however, decreased if the number of carbon atom was further increased.

### Test Example 4

### Potentiating Effect of isoamyl Gallate for the Antibacterial Activity of other β-lactams (by the Agar Plate Dilution Method)

Since isoamyl gallate showed excellent effect of the combination use, MIC was determined as described in example 2 and the effect of the combination use of isoamyl gallate and other β-lactams was studied.

TABLE 4

| | | Penicillin G | | Cefoxitin | | Ampicillin | | Cefapirin | |
|---|---|---|---|---|---|---|---|---|---|
| | | (mg/ml) | | (mg/ml) | | (mg/ml) | | (mg/ml) | |
| | | | Isoamyl | | Isoamyl | | Isoamyl | | Isoamyl |
| | | | gallate | | gallate | | gallate | | gallate |
| Strain No. | | alone | 25 mg/ml | alone | 25 mg/ml | alone | 25 mg/ml | alone | 25 mg/ml |
| MRSA | 1 | 64 | 8 | >256 | 64 | 32 | 8 | 128 | 16 |
| | 2 | 64 | 0.5 | 256 | 4 | 64 | 0.5 | 128 | 0.06 |
| | 3 | 32 | 0.25 | 256 | 0.25 | 16 | 1 | 64 | 0.016 |
| | 4 | 32 | 4 | >256 | 16 | 32 | 4 | 64 | 4 |
| | 5 | 64 | 2 | >256 | 16 | 32 | 2 | 64 | 4 |
| | 6 | 32 | 1 | 256 | 4 | 32 | 2 | 64 | 0.5 |
| | 7 | 32 | 8 | >256 | 4 | 32 | 4 | 64 | 0.016 |
| | 8 | 32 | 0.063 | 256 | 0.5 | 16 | 0.25 | 64 | 0.016 |
| | 9 | 32 | 2 | >256 | 4 | 32 | 4 | 64 | 0.5 |
| | 10 | 32 | 0.5 | 64 | 4 | 32 | 0.5 | 64 | 0.063 |
| | 12 | 32 | 4 | >256 | 32 | 32 | 4 | 128 | 4 |
| | 16 | 32 | 8 | >256 | 16 | 32 | 8 | 64 | 4 |
| | 17 | 32 | 1 | 256 | 4 | 32 | 1 | 64 | 0.5 |
| | 18 | 32 | 4 | >256 | 16 | 32 | 4 | 64 | 4 |
| | 20 | 16 | 0.063 | 32 | 2 | 8 | 0.25 | 16 | 0.063 |
| | 21 | 16 | 0.25 | 64 | 2 | 8 | 0.5 | 32 | 0.5 |
| | 22 | 16 | 0.25 | 64 | 4 | 8 | 0.5 | 16 | 0.5 |
| | 24 | 8 | 0.25 | 8 | 0.5 | 16 | 0.5 | 0.25 | 0.063 |
| | COL | 32 | 1 | >256 | 16 | 32 | 2 | 64 | 1 |
| MSSA | 1003 | 0.13 | 0.063 | 4 | 2 | 0.5 | 0.25 | 0.5 | 0.063 |
| | 1010 | 0.13 | 0.063 | 4 | 2 | 0.5 | 0.13 | 0.25 | 0.063 |
| | 1020 | 0.13 | 0.031 | 4 | 0.25 | 0.25 | 0.063 | 0.25 | 0.063 |
| | 1023 | 4 | 0.25 | 8 | 2 | 8 | 0.5 | 0.5 | 0.063 |
| | 1029 | 8 | 0.25 | 8 | 2 | 16 | 0.5 | 0.5 | 0.063 |
| | 1032 | 0.063 | 0.063 | 2 | 0.5 | 0.13 | 0.13 | 0.063 | 0.063 |
| ATCC | 6538 | 0.063 | <0.016 | 2 | 0.031 | 0.25 | 0.031 | 0.063 | 0.016 |

| | | | | | | | | |
|---|---|---|---|---|---|---|---|---|
| RN4220 | 0.031 | 0.031 | 2 | 0.25 | 0.063 | 0.063 | 0.063 | 0.063 |

As shown in Table 4, isoamyl gallate showed excellent effects of the combination use with penicillin G, cefoxitin, ampicillin, and cefapirin.

### Test Example 5

### Potentiating Effect of Galloyl Quinic Acid Derivatives for the Antibacterial Activity of Oxacillin (by the Agar Plate Dilution Method)

It was confirmed that the known substances shown below were contained in “Tara” and their MICs were determined as described in example 2 with respect to the effects of the combination use with oxacillin

TABLE 5

| | R₁ | R₂ | R₃ | R₄ |
|---|---|---|---|---|
| (1) Methyl 4,5-digalloylquinate | H | G | G | Me |
| (2) Methyl 3,4,5-trigalloylquinate | G | G | G | Me |
| (3) 3,4,5-trigalloylquinic acid | G | G | G | H |
| (4) 3,4-digalloylquinic acid | G | G | H | H |

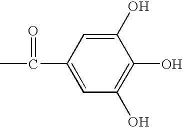

G:

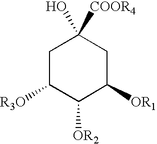

Oxacillin MIC (μg/ml)

| | | | 1 | | | 2 | | | 3 | | | 4 | |
|---|---|---|---|---|---|---|---|---|---|---|---|---|---|
| | | 12.5 | 25 | 50 | 12.5 | 25 | 50 | 12.5 | 25 | 50 | 12.5 | 25 | 50 |
| Strain No. | — | μg/ml | μg/ml | μg/ml | μg/ml | μg/ml | μg/ml | μg/ml | μg/ml | μg/ml | μg/ml | μg/ml | μg/ml |
| MRSA 1 | >256 | 128 | 128 | 128 | >256 | >256 | >256 | 128 | 32 | 64 | >256 | >256 | >256 |
| 2 | 256 | 2 | 2 | 2 | 256 | 32 | 32 | 2 | <0.5 | <0.5 | >256 | 256 | 256 |
| 3 | 128 | <0.5 | 0.5 | 1 | 64 | 32 | 32 | 4 | <0.5 | 1 | 256 | 256 | 128 |
| 4 | 256 | 128 | 128 | 32 | 256 | 256 | 256 | 32 | 4 | 2 | >256 | >256 | >256 |
| 5 | 256 | 128 | 32 | 64 | >256 | 256 | 256 | 64 | 8 | 8 | >256 | ≅256 | >256 |
| 6 | >256 | 2 | 2 | 4 | 128 | 32 | 64 | 4 | 2 | 4 | 256 | 256 | 128 |
| 7 | >256 | 32 | 16 | 16 | 256 | 128 | 64 | 16 | 4 | 2 | >256 | >256 | >256 |
| 8 | 128 | <0.5 | 16 | 1 | 64 | 64 | 64 | 8 | 4 | 8 | 256 | 128 | 128 |
| 9 | 256 | 16 | 16 | 8 | >256 | 256 | 128 | 8 | 2 | 2 | >256 | >256 | >256 |
| 10 | 128 | 4 | 8 | 8 | 64 | 64 | 64 | 4 | 2 | 4 | 128 | 128 | 128 |
| 12 | >256 | 16 | 16 | 16 | 256 | 128 | 256 | 8 | 4 | 4 | >256 | >256 | >256 |
| 16 | 256 | 128 | 128 | 64 | >255 | 256 | 256 | 32 | 8 | 8 | >256 | >256 | >256 |
| 17 | 256 | 32 | 32 | 4 | 128 | 256 | 256 | 4 | 8 | 4 | 256 | 256 | 256 |
| 18 | >256 | 128 | 128 | 32 | 256 | 128 | 128 | 32 | 4 | 4 | >256 | >256 | >256 |
| 20 | 64 | <0.5 | 0.25 | <0.5 | 8 | 2 | 4 | <0.5 | <0.5 | <0.5 | 32 | 16 | 16 |
| 21 | 64 | <0.5 | 0.25 | 1 | 32 | 8 | 16 | 1 | <0.5 | <0.5 | 128 | 128 | 64 |
| 22 | 32 | 1 | 0.5 | 1 | 32 | 16 | 32 | 1 | <0.5 | 1 | 64 | 64 | 64 |
| COL | 256 | 8 | 32 | 2 | 256 | 128 | 128 | 8 | 4 | 4 | >256 | >256 | >256 |
| MSSA 1003 | 0.25 | <0.5 | 0.25 | <0.5 | <0.5 | <0.5 | <0.5 | <0.5 | <0.5 | <0.5 | <0.5 | <0.5 | <0.5 |
| 1010 | 0.25 | <0.5 | 0.5 | <0.5 | <0.5 | <0.5 | <0.5 | <0.5 | <0.5 | <0.5 | <0.5 | <0.5 | <0.5 |
| 1032 | 0.13 | <0.5 | 0.25 | <0.5 | <0.5 | <0.5 | <0.5 | <0.5 | <0.5 | <0.5 | <0.5 | <0.5 | <0.5 |
| ATCC 6538 | 0.063 | <0.5 | <0.063 | <0.5 | <0.5 | <0.5 | <0.5 | <0.5 | <0.5 | <0.5 | <0.5 | <0.5 | <0.5 |
| RN4220 | 0.13 | <0.5 | 0.125 | <0.5 | <0.5 | <0.5 | <0.5 | <0.5 | <0.5 | <0.5 | <0.5 | <0.5 | <0.5 |

(1): Methyl 4,5-digalloylquinate
(2): Methyl 3,4,5-trigalloylquinate
(3): 3,4,5-trigalloylquinic acid
(4): 3,4-digalloylquinic acid

Result

With respect to the effect of combination use with oxacillin, 3,4,5-trigalloylquinic acid was most potent, and Methyl 4,5-digalloylquinate follows; Methyl 3,4,5-trigalloylquinate and 3,4-digalloylquinic acid showed weak effects.

### Test Example 6

### Potentiating Effect of Various Gallates for Antibacterial Activity of Oxacillin [Measurement of MIC (by Broth Microdilution Method) and FIC]

According to the broth microdilution method defined by Japan Society of Chemotherapy, MIC was determined. CAMHA was prepared by adding 50 mg/l of Ca-ion, 25 mg/l of Mg-ion, and 2% of NaCl to MHB (Mueller-Hinton Broth), and it was used as a medium for use in the measurement of sensitivity. In 96-well microplates 100 μl of the medium for use in the measurement of sensitivity containing drugs was dispensed. The bacterial liquid was incubated at 37° C. overnight, diluted with saline and added to the plate so that the final concentration of the bacteria was 5×10⁵ CFU/well. After incubation at 37° C. for 24 hr, MIC was determined from the presence or absence of the bacterial development. FIC (fractional inhibitory concentration) was calculated from the result and the potency of the effect of the combination use was evaluated. The results were shown in Table 6.

TABLE 6

| | Oxacillin | | | |
|---|---|---|---|---|
| | MRSA COL | | MRSA Strain No. 5 | |
| Compound | MIC (mg/ml) | FIC value | MIC (mg/ml) | FIC value |
| Methyl gallate | >1600 | — | >1600 | — |
| Ethyl gallate | 400 | 0.125 | 400 | 0.156 |
| Propyl gallate | 100 | 0.258 | 100 | 0.258 |
| Butyl gallate | 100 | 0.129 | 50 | 0.254 |
| Isobutyl gallate | 50 | 0.129 | 50 | 0.266 |
| Pentyl gallate | 25 | 0.375 | 12.5 | 0.563 |
| Isoamyl gallate | 25 | 0.25 | 25 | 0.313 |
| Hexyl gallate | 6.25 | 0.5 | 6.25 | 0.502 |
| Heptyl gallate | 6.25 | 0.563 | 3.13 | 0.75 |
| Octyl gallate | 12.5 | 0.252 | 3.13 | 0.75 |
| Nonyl gallate | 6.25 | 0.625 | 12.5 | 0.502 |
| Decyl gallate | 12.5 | 0.258 | 6.25 | 0.508 |
| Undecyl gallate | 12.5 | 0.313 | 6.25 | 0.251 |
| Dodecyl gallate | 25 | 0.531 | 50 | 0.375 |
| Catechin | — | <0.25 | 100 | 0.064 |
| gallate | | | | |
| Gallocatechin | 100 | 0.281 | 100 | 0.188 |
| gallate | | | | |
| Epicatechin | 50 | 0.127 | 25 | 0.188 |
| gallate | | | | |

Result

As shown in Table 6, the gallates were found to have the antibacterial activity and the effects of the combination use with oxacillin in the tested MRSA, i.e, COL Strain and Strain No. 5. In Table 6, MIC means those of the various gallates themselves and the antibacterial activities of octyl gallate, nonyl gallate, decyl gallate, and undecyl gallate were most potent when used alone. On the other hand, butyl gallate and isobutyl gallate were found to have the most potent effect of the combination use by referring to their FIC values, which show the effect of the combination use with oxacillin. Also, catechin gallate, gallocatechin gallate, and epicatechin gallate showed the more potent effects of the combination use, especially catechin gallate was most potent.

### EXAMPLES

### Example 1

### Preparation of “Tara” Extract

1 kg of “Tara” was extracted with 10 parts of 50% EtOH at 60° C. for 2 hr. After the extraction, it was filtered and the filtrate was condensed to about a quarter at 60° C. Ethyl acetate in a equal amount of the residue was added and the organic layer was separated, condensed to a dried residue, which was purified by column chromatography eluted with n-hexane-ethyl acetate. The fraction was condensed to a dried residue to afford about 2.5 g of “Tara” extract.

### Example 2

### Tablet

According to the conventional process, 50 mg of isoamyl gallate, 50 mg of oxacillin, 1 g of lactose, 300 mg of starch, 50 mg of methylcellulose and 30 mg of talc are formulated to give ten tablets which are then coated with sucrose.

### Example 3-18

### Tablet

According to the example 2 wherein isoamyl gallate is replaced with methyl gallate, ethyl gallate, n-propyl gallate, n-butyl gallate, n-pentyl gallate, n-hexyl gallate, n-heptyl gallate, n-octyl gallate, n-nonyl gallate, n-decyl gallate, n-undecyl gallate, n-lauryl gallate, isobutyl gallate, catechin gallate, gallocatechin gallate, or epicatechin gallate, tablets are prepared.

### Example 19

### Injectable

A sterile mixture containing 500 mg of methyl gallate and 500 mg of oxacillin are placed in a sterilized vial which is then sealed. Upon use, this mixture is dissolved in saline to give an injectable.

### Example 20-27

### Injectable

According to example 19, wherein methyl gallate is replaced with ethyl gallate, n-propyl gallate, n-butyl gallate, n-pentyl gallate, isobutyl gallate, catechin gallate, gallocatechin gallate, or epicatechin gallate, an injectable is prepared.

### Example 28

### Disinfectant

A disinfectant prepared from isoamyl gallate, oxacillin, ethanol, and sterilized water is shown. These ingredients are mixed with the ratio below;

| | | |
|---|---|---|
| isoamyl gallate | 25 | mg |
| oxacillin | 10 | mg |
| ethanol | 500 | ml |
| sterilized water | 500 | ml |

### Example 29

### Functional Food

10 L of 50% EtOH is added to 1 kg of “Tara” pod and thermally extracted. The extraction is filtered, and the filtrate is condensed to a suitable amount, spray-dried to give an essence. Carbohydrate such as sugar, honey, and liquid sugar syrup is added to the essence, and cookies etc. are prepared. Also, the essence is formulated to a tablet or powder to afford functional foods.

### INDUSTRIAL APPLICABILITY

The multivalent phenol derivatives and/or “Tara” extract of the invention can potentiate the antibacterial effect of β-lactam antibiotics and other antibacterial drug when used in the combination with these antibiotics and antibacterial drug. Furthermore, they can reduce the amount of β-lactams or antibacterial drugs clinically used, and chances for the bacteria to acquire resistance to β-lactam antibiotics in advance. Accordingly, the invention is used as a pharmaceutical composition for the treatment of infections with a drug resistant bacteria utilizing the characteristic that the multivalent phenol derivatives and/or “Tara” extracts potentiate the antibacterial effect of β-lactam antibiotics; the invention is also used as a disinfectant or functional food comprising the multivalent phenol derivatives and/or “Tara” extracts, which has the antibacterial activity for the drug resistant bacteria.

1-13. (canceled)

14. An enhancer of β-lactam antibiotics comprising a multivalent phenol derivative of a formula (III); 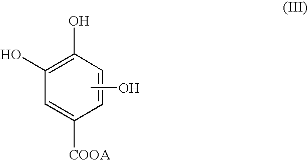
wherein A is a lower alkyl or a group of a formula (IV); 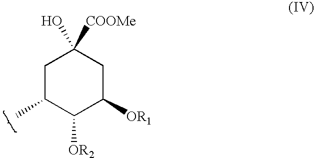
wherein R₁ is H or a galloyl group, R₂ is a galloyl group,
or a pharmaceutically acceptable salt thereof and/or extract from “Tara”.

15. An enhancer of β-lactam antibiotics comprising a multivalent phenol derivative of a formula (V); 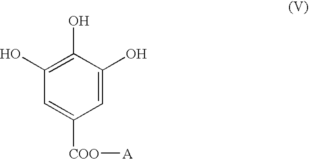
wherein A is a lower alkyl or a group of a formula (IV); 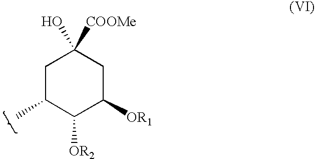
wherein R₁ is H or a galloyl group, R₂ is a galloyl group,
or a pharmaceutically acceptable salt thereof and/or extract from “Tara”.

16. The enhancer according to claim 14 or claim 15 wherein the said β-lactam antibiotics are selected from ampicillin, benzylpenicillin, phenethicillin, methicillin, oxacillin, carbenicillin, cefapirin, cefradine, cefuroxime, cefoxitin, cefotaxime, panipenem and mixtures thereof.

17. The enhancer according to claim 14 or claim 15 comprising a multivalent phenol derivative of the formula (V)′ 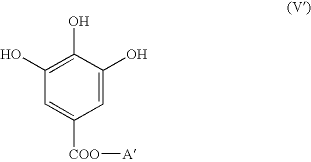
wherein A′ is a lower alkyl group, or a pharmaceutically acceptable salt thereof as an active ingredient.

18. The enhancer according to claim 14 or claim 15 comprising methyl 4,5-digalloylquinate and/or methyl 3,4,5-trigalloylquinate as an active ingredient.

19. The enhancer according to claim 14 or claim 15 comprising extract from “Tara” as an active ingredient.

20. A pharmaceutical composition for a therapy of an infection with a drug resistant bacterium comprising the enhancer according to claim 14 or claim 15 and a β-lactam antibiotic.

21. A method for treating an infection with a drug resistant bacterium characterized by administering an effective amount of a β-lactam antibiotic and an enhancer according to claim 14 or claim 15.

22. (canceled)

23. A disinfectant for a drug resistant bacterium comprising a β-lactam antibiotic and an enhancer according to claim 14 or claim 15.
